(19) United States
(12) Patent Application Publication
(10) Pub. No.: US 2005/0118236 A1
(43) Pub. Date: Jun. 2, 2005

(54) Bioactive, resorbable scaffolds for tissue engineering

(75) Inventors: Qing-Qing Qiu, Kingston (CA); Charles S. Cohen, Gladwyne, PA (US); Paul Ducheyne, Rosemont, PA (US)

Correspondence Address: WOODCOCK WASHBURN LLP, ONE LIBERTY PLACE, 46TH FLOOR, 1650 MARKET STREET, PHILADELPHIA, PA, 19103, US

(73) Assignee: Gentis Inc.

(21) Appl. No.: 10/721,028

(22) Filed: Nov. 24, 2003

Related U.S. Application Data
(60) Provisional application No. 60/430,529, filed on Dec. 3, 2002.

(51) Int. Cl. A61K 45/00; A61K 9/70

(52) U.S. Cl. 424/443; 424/93.7; 442/123

(57) ABSTRACT

Flexible, bioactive glass meshes and scaffolds made therefrom are provided. The meshes comprise interwoven bioactive glass fibers that can be coated with resorbable polymers. Meshes can also be woven from glass fibers and resorbable polymers. Scaffolds can be constructed by a plurality of meshes, which can have varying porosities to create porosity gradients in the scaffold. Methods of making scaffolds are provided which can comprise pulling bioactive glass fibers, winding the fibers, forming the fibers into bundles, coating the fibers with a resorbable polymer, and creating a biaxial weave with the bundles. Soft tissue engineering methods are also provided for creating scaffolds for incubating cells such as fibroblasts and chondroblasts. Meshes and scaffolds are suitable for tissue engineering, such as bone tissue engineering and cartilage tissue engineering.

### CROSS-REFERENCE TO RELATED APPLICATION

This claims priority from U.S. Provisional Application No. 60/430,529 filed Dec. 3, 2002.

### FIELD OF THE INVENTION

The present invention relates to tissue engineering applications and bioactive glass/polymer scaffolds for the repair of cartilage and bony defects.

### BACKGROUND

Over 16 million people in the US suffer from severe joint pain and related dysfunction as a result of injury or osteoarthritis. The biological basis of joint problems is the deterioration of articular cartilage. There are 500,000 cartilage surgeries in the US alone and about 1 million cases worldwide.

Current treatments for articular defects have limited success in that they are deficient in long-term repair or have unacceptable side effects. The treatments such as injecting lubricating fluids to relieve pain, abrasion arthroscopy, subchondral bone drilling and microfracture typically result in fibrocartilage filling the defect site. Autograft procedures, such as Mosaicplasty (Hangody L, Feczko P, Bartha L, Bodo G, Kish G (2001) Mosaicplasty for the treatment of articular defects of the knee and ankle. Clin Orthop S391:S328-36) and Osteochondral Autograft Transfer System (OATS) (Attmanspacher W, Dittrich V, Stedtfeld H W (2000) Experiences with anthroscopic therapy of chondral and osteochondral defects of the knee joint with OATS (Osteochondral Autograft Transfer System). Zentralbl Chir 125:494-9), that remove an osteochondral plug from a non-load bearing area and graft it into the defect site, require additional time to acquire the donor tissue and result in donor site morbidity and pain. Allogeneic transplantation of osteochondral grafts has had clinical success, but supply is limited and has a risk of infection.

A typical current tissue engineering approach to cartilage repair requires obtaining cells from a cartilage biopsy, which requires an additional surgical procedure if informed consent is not obtained from the patient prior to the arthroscopic exploratory procedure. In addition, the cell source is limited. One approach for repairing cartilage starts with cells that are easily obtained from skin tissue (Nicoll S B, Wedrychowska A, Smith N R, Bhatnager R S (2001) Modulation of proteoglyean and collagen profiles in human dermal fibroblasts by high density micromass culture and treatment with lactic acid suggests change to a chondrogenic phenotype. Conn Tiss Res 42, 59-69). This technology uses micromass cultures. In U.S. Pat. No. 6,197,586, Bhatnagar and Nicoll discuss “Chondrocyte-like cells useful for tissue engineering and methods” and provide treatments of fibroblast cells “with a chemical inhibitor of protein kinase C such as staurosporine, in conjunction with functionally hypoxic micromass culture so as to be induced into chondrogenic differentiation.”

In tissue engineering, one uses cells, biological molecules, and carrier materials (i.e. scaffolds) to aid the healing, repair, and regeneration of tissues and organs. For example, Ma and Zhang discuss “Preparation and morphology of poly(α-hydroxyl acids)/hydroxyapatite porous composites for bone-tissue engineering” making scaffolds of porous calcium phosphates. In U.S. Pat. Nos. 5,643,789; 5,676,720; 5,811,302; and 5,648,301, Ducheyne, El-Ghannam, and Shapiro discuss porous bioactive glasses and methods for making and conditioning them. The scaffolds of Ma and Ducheyne are rigid and cannot be shaped sufficiently at the time of surgery. Hence, neither the scaffolds of Ma nor Ducheyne could be made to contour an organ or tissues.

Other physical forms of bioactive glass also have limited application in the repair of cartilage and soft tissue due to, for example, their rigidity, low porosity, and limited resorbability, for example, glass granules discussed by U.S. Pat. No. 5,658,332 (Schepers et al.), bioactive glass fibers in a non-resorbable polymer matrix discussed by U.S. Pat. No. 5,468,544 (Marcolongo et al.) and U.S. Pat. No. 5,721,049 (Marcolongo et al.). Furthermore, mechanical behaviors exhibited by bioactive glass fibers by themselves discussed by U.S. Pat. No. 5,645,934 (Marcolongo et al.) and microspheres of PLA and glass powder discussed by U.S. Pat. No. 6,328,990 (Qiu et al.) are not satisfactory for cartilage repair.

In tissue engineering, it is desirable to use scaffolds that follow the contours of the organ or tissue to be treated. Such is, for instance, the case for treatment of cartilage pathology and injury (e.g., lesions). Some products and methods that have been used relied on the use of resorbable polymers, primarily synthetic materials, such as polylactic acid polymer or polylactide (PLA), polyglycolide (PGA), and polylactide-co-glycolide (PLGA), and biologic scaffold, such as collagen. These scaffolds are usually combined with cells.

The use of cells creates concerns of expense, morbidity, and risk for disease transmission. If cells are taken from a patient, then there is often morbidity associated with the donor site. If cells come from a donor, then there is often the latent fear for transmission of known or unknown pathogens. Using collagen with cells presents a limitation in lacking sufficient mechanical properties. Furthermore, collagen is typically supplied by a bovine source, which evokes the potential for disease transmission. When collagen is supplied by recombinant techniques using human collagen molecules, the product is very expensive.

As mentioned above, low porosity of a scaffold limits the usefulness of such a scaffold for cartilage and other soft tissue repair. Although high porosity is desirable, it is also desirable to utilize a gradient in porosity for treating lesions and bony defects. The use of a gradient in porosity has been suggested in the context of bone repair (P. Ducheyne, P. De Meester, E. Aemoudt; Isostatically compacted metal fiber porous coatings for bone ingrowth, Powder Metallurgy Int. 11:115-119, 1979). In addition, Therics (Princeton, N.J.) has technology, TheriForm™ that allows to make products with gradients in porosity. (J. K. Sherwood, S. L. Riley, R. Palazzolo, S. C. Brown, D. C. Monkhouse, M. Coates, L. G. Griffith, L. K. Landeen, A. Ratcliffe, A three-dimensional osteochondral composite scaffold for articular cartilage repair, 23 Biomaterials 4739-4751 (2002)). However, Therics' TheriForm™ is not easily applied to composites that include a ceramic component. Furthermore, TheriForm™ will not be flexible if a high amount of TCP is used and thus will not be adaptable to cartilage contours.

There is a great need for scaffolds and methods to provide scaffolds and methods for transplanting cartilage to a defect. It is an intent in the present invention to culture cells on a three-dimensional bioactive, porous, and resorbable scaffold. A further objective is to develop three-dimensional scaffolds that support cartilage formation and have a reliable fixation into a defect and integration with the surrounding tissues. In addition, for defects in articular locations with substantial curvature, the scaffold should allow the tissue-engineered constructs to have appropriate topography. Other features of the scaffold include a highly porous and lactate-rich region for promoting cartilage regeneration, and a bioactive matrix that stimulates tissue formation and repair.

### SUMMARY OF THE INVENTION

The present invention provides composite synthetic/biologic scaffolds which are viable for tissue engineering of cartilage in vitro and transplanting the cartilage to a defect. The present invention also provides bioactive, flexible, bioactive glass weaves and scaffolds with high porosity. In addition, the present invention provides method and scaffolds for developing cartilage tissue in vitro. Methods and scaffolds according to the present invention are suitable for many aspects of tissue engineering, including but not limited to bone tissue engineering and cartilage tissue engineering.

In an aspect of the present invention, flexible, bioactive glass meshes comprising interwoven bioactive glass fibers coated with a resorbable polymer is provided. The meshes can comprise a porosity of between about 25% and 95%. The glass fibers can be coated with any suitable resorbable polymer, for example, polylactic acid polymers (PLA) and/or poly-glycolic acid polymers and/or their copolymers.

Another aspect of the present invention provides flexible, bioactive meshes comprising glass fibers and first resorbable polymer fibers wherein the glass fibers are interwoven with the polymer fibers. The glass fibers themselves can be coated with a second resorbable polymer. The second resorbable polymer can be the same as or different from the first resorbable polymer. The glass fibers can be woven perpendicularly to the polymer fibers. Further, a portion of the polymer fibers along with the glass fibers can be woven perpendicularly to another portion of the polymer fibers.

An additional embodiment includes flexible, bioactive scaffolds comprising a plurality of bioactive meshes which comprise interwoven bioactive fibers coated with a resorbable polymer. The plurality of bioactive meshes can then be attached by methods not limited to lamination, stitching, and chemical treatment, (e.g., using alcohol and/or solvent for two-dimensional and three-dimensional coherence).

Other aspects of the present invention include scaffolds having a gradient in porosity and methods for making them. Scaffolds in accordance with the present invention can comprise a cartilage region or a bone region or both. Also, scaffolds may include a non-calcified tissue region. In the cartilage region, a porosity of between about 40% and 95% is desirable, preferably more than 60%, and even more preferably more than 80% is desirable. A porosity greater than 25% is desirable for the region of the scaffold that goes into bone, preferably between about 25% and 80%. A porosity of between about 25% and 90% may be desirable in the non-calcified region. A gradient in porosity is achieved through the weaving and subsequent three dimensional assembly of the weaves which creates a three-dimensional structure with layers of weaves in which the subsequent layers have different weaving characteristics and therefore different porosity (and also pore size) characteristics.

The present invention is based in part on the unexpected finding that bioactive glass stimulates chondrocyte function.

The degree of porosity and resorbability of scaffolds impacts the suitability of a scaffold for repair of soft tissue. With respect to porosity, as the bioactive scaffold can be used by itself, without the need to seed it with cells prior to implantation, a large porosity (for example, a porosity that exceeds 60%) is useful, such that cells can proliferate from the tissues supporting the cartilage in joints. Even if the scaffolds are seeded with cells prior to surgery, the large porosity would make for an efficient distribution of the cells throughout the scaffold. Large porosity is also desirable as it allows the achievement of mechanical properties very similar to those of the tissue that needs to be treated, i.e., elastic properties.

In terms of resorbability, whereas it is acceptable that elastic properties of the engineered cartilage immediately postoperatively (that is, upon insertion of the scaffold) are not the same as those of native cartilage, in the medium and long term (i.e., from about 6 to 12 months on) the repaired site should have properties equivalent to those of native cartilage. Thus, it is desirable that the material of the scaffold be resorbable.

Some aspects of the present invention include bioactive, flexible, bioactive glass weaves with high porosity. As indicated above, bioactive glass stimulates chondrocyte function (“bioactive”). Fine wires of bioactive glass are resorbable. Weaving bundles of glass fibers creates a scaffold having high porosity. Coating glass fibers with PLA results in resorbable material, which improves the manufacturability of the glass fibers (the glass fibers are difficult to be woven by themselves); does not adversely affect the bioactivity of the glass. Resorption of PLA produces a microenvironment that is beneficial for chondrocyte function: the degradation of the PLA produces lactate, which is known to be present in the microenvironment of chondrocytes, and appears to have a beneficial effect on chondrocyte function in vitro (U.S. Pat. No. 6,197,586 to Nicoll and Bhatnagar).

Further aspects of the present invention include the use of bioactive glass for treating cartilage lesions. In one method, a flexible, bioactive glass scaffold is provided, chondrocyte-like cells are seeded onto the glass scaffold, and the glass scaffold is implanted into a mammal.

In one embodiment of the present invention, a porous structure comprising bioactive glass fiber scaffolds is used for the treatment of lesions in which contouring the tissue and or organ is important.

Methods for making such scaffolds are also provided. In one method in accordance with the present invention, bioactive glass fibers are pulled, wound, coated with a resorbable polymer to form bundles, and then the bundles are used to create a biaxial weave. In addition, a plurality of biaxial weaves can be used to create a three-dimensional scaffold. Furthermore, the plurality of biaxial weaves can have differing porosities thereby creating a porosity gradient.

In another method in accordance with the present invention, bioactive glass fibers are pulled, wound, and formed into bundles, the bundles are then coated with a resorbable polymer and used to create a biaxial weave. In addition, a plurality of biaxial weaves can be used to create a three-dimensional scaffold. Furthermore, the plurality of biaxial weaves can have differing porosities thereby creating a porosity gradient.

Scaffolds in accordance with the present invention can also be used as a carrier for the delivery of cells and molecules into an in vivo site.

In yet another aspect of the present invention, a method of engineering soft tissue is provided comprising creating a biaxial weave comprising interwoven glass fibers, creating a flexible bioactive glass scaffold comprising the glass fibers, seeding fibroblasts onto the glass scaffold, and incubating the fibroblasts. Other methods include creating a biaxial weave comprising interwoven glass fibers, creating a flexible bioactive glass scaffold comprising the glass fibers, seeding chondroblasts onto the glass scaffold, and incubating the chondroblasts.

Below are several examples of specific embodiments for carrying out the present invention. The examples are offered for illustrative purposes only, and are not intended to limit the scope of the present invention in any way.

### BRIEF DESCRIPTION OF THE DRAWINGS

FIGS. 1A and 1B are optical micrographs of a PLA-coated bioglass weave after single needle punching according to the present invention. Original magnification: (A) 10×; (B) 30×.

FIG. 2A shows osteochondral defect 1 month after SHAM surgery. Toluidine Blue Stain (Original magnification 20×).

FIG. 2B shows osteochondral defect 3 months after surgical placement of a device in accordance with the present invention. Safranin O stain (Original magnification 20×).

FIG. 3A shows a two-dimensional fabric woven with glass and polymer yarns in accordance with the present invention.

FIG. 3B shows a three-dimensional fabric woven with glass and polymer yarns in accordance with the present invention.

FIG. 4 shows a porous scaffold of a higher pore size than FIG. 3A in accordance with the present invention.

### EXAMPLE 1

### Preparation of Scaffold

Porous and bioactive scaffolds were fabricated with fine bioactive glass fibers using a weaving method. The composition of the bioactive glass and the fabrication of the glass fibers were described previously (Marcolongo M, Ducheyne P, LaCourse W C. (1997) Surface reaction layer formation in vitro on a bioactive glass fiber/polymeric composite. J Biomed Mater Res. 1997 Dec. 5;37(3):440-8 and Marcolongo M, Ducheyne P, Garino J, Schepers E. (1998) Bioactive glass fiber/polymeric composites bond to bone tissue. J Biomed Mater Res. 1998 January;39(1):161-70). The diameter range of a single glass fiber is 15-25 μm.

Porous scaffolds were fabricated with glass bundles. Briefly, a 1 mm wide glass bundle consisting of glass fibers (Glass International, Covina, Calif.) was brushed with 10% polylactic acid (PLA) (MW 200,000, Polyscience) solution in chloroform. The bundles were manually woven into a simple biaxial pattern. The woven scaffold was cleaned in alcohol (isopropanol), and brushed with 10% PLA solution on both sizes followed by drying in air. The micropore size and the distance between bundles were in the range 150-200 μm and 400-800 μm respectively.

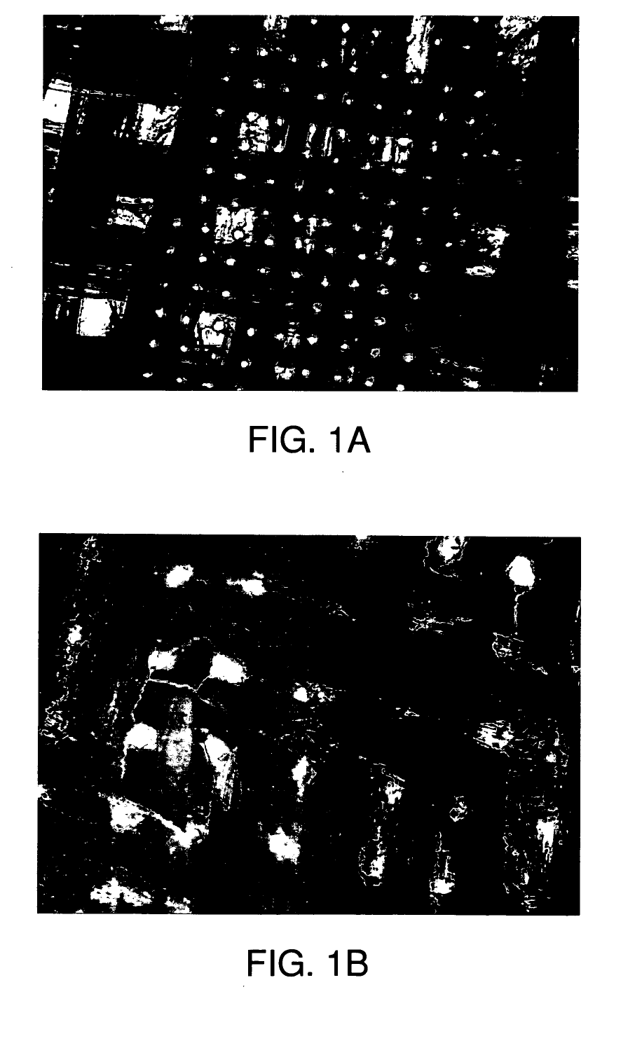

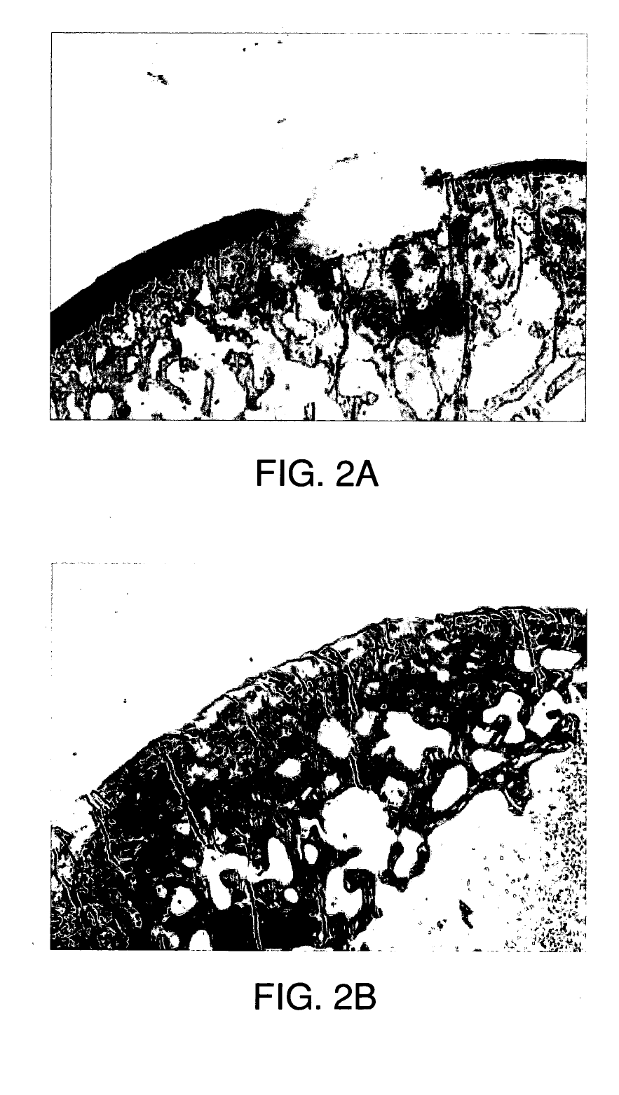

A regular pattern of micropores resulted from needle punching. By using fixed size punching needles and an μm-scale X-Y displacement, holes of about 200 μm at a distance of about 400 μm were made in the PLA-coated weave (FIG. 1A and B). Discs of 3.5 mm in diameter were punched from the weave. The discs were cleaned in alcohol, sterilized, conditioned in buffered solution, dried and packaged in sterile pouches for use in the animal implant study.

### EXAMPLE 2

### In Vivo Scaffold Absorption

The scaffolds fabricated using the method described in Example 1 were implanted in the patellar groove of rabbits for 4 and 12 months. Briefly, twelve New Zealand white rabbits were used. Two defects, 3.5 mm in diameter and 0.5 mm in depth, were created in the rabbit left and right trochlear groove by hand drilling. Four experimental groups were used: control (defect only, without implant), or defects filled with implants prepared according to any of three treatment schemes: woven scaffolds without subsequent treatment to transform the glass surface (unconditioned scaffold, or scaffold A), or woven scaffolds treated in either serum free or serum containing solutions (conditioned scaffolds or scaffolds B or C). The implants were retrieved at 1-month (n=6) and 3-month (n=6) and their histological evaluation was carried out by Skeletech, Bothell, Wash.

Histological evaluation (Skeletech (2001) Qualitative evaluation on histological sections of full thickness cartilage defect from rabbit trochlear grooves implanted with a biosynthetic cartilage replacement material. SkeleTech Inc.):

The tissue blocks of interest were processed un-decalcified by infiltration with methyl methacrylate using a cold embedding method to preserve heat labile components of the implant. Once embedded, two 5-10 μm vertical sections were prepared from each block through the center of the defect in a sample. One of the sections was stained with safranin O, the other one with toluidine blue.

In this study, defects were created into the subchondral bone to breach the tidemarks and thereby cause a bleeding into the defect. Any inflammation observed was at most mild to moderate. The best averaged healing found at the 1-month timepoint is about 50% in the defects which received no scaffold. This may be the result of the fact that young rabbits have an excellent ability to heal a defect that breached the subchondral bone region. The overall scores increased modestly to 75% in this type of defect by 3-month post implantation. However, the largest increase (between the two timepoints) was found in defects which received the scaffold that was unconditioned (Scaffold A). This is followed by the defects which received scaffolds that were conditioned without serum (Scaffold B) and the defects which received the scaffolds that were conditioned with serum (Scaffold C). Because of the difficulty in healing full thickness articular cartilage, the healing progression in defects which received either Scaffold A or B is considered to have biological significance within the study period. The implication is based on the need for an initial host cells/tissue interaction with the implanted scaffold, which is then followed by the ingrowth of chondrogenic/osteogenic cells and the formation of reparative cartilage/bone in the defects. This is supported by the Safranin O staining on GAG content of the reparative cartilage. A trend of increasing GAG staining intensity was present between the two timepoints.

The qualitative evaluation indicates that two full thickness cartilage defects can be made on one femoral condyle and two types of scaffolds can be used in adjacent defects without cross-interaction between the sites. Since the control sites can heal by themselves, this model cannot differentiate the effectiveness of these various scaffolds. However, this study demonstrated that the scaffold are fully resorbed and are fully compatible with surrounding bone and cartilage tissue. Comparing the healing of the defects treated with three different scaffolds, the results indicated that both scaffolds A and B have the potential for being effective in achieving cartilage repair in osteochondral defects.

### EXAMPLE 3

### Fabrication and Weaving of Glass Bundles

A bundle of glass filaments having a diameter of approximately 100-350 μm is desirable. In contrast, the usefulness of a bundle of thick glass having a similar diameter is limited because it is brittle and inflexible.

Bioactive glass fibers of 15-25 μm in diameter are pulled from a ˜1 mm aperture of a bushing at melting temperature of 1140° C. while being wound on a drum of 30.48 cm in diameter rotating at 275 rpm. Because the bioactive glass fibers are known to be fragile and difficult to handle, they are coated with polylactic acid (PLA) polymer dissolved in chloroform (2% w/v) to form bundles of 100-350 μm in diameter to enhance their handling properties. The PLA polymer serves as a binder for the glass filaments in the bundle. Biaxial weave is made with the glass bundles.

In two-dimensional weaving, almost all patterns that can be done with polymer yarns can also be done with glass bundles. Specific procedures for different weaving patterns are numerous and widely available to those skilled in the art. An exemplary, but by no means exhaustive compilation of weaving patterns is provided in Textiles: Fiber to Fabric by M. David Potter, Bernard P. Corbman. McGraw-Hill Book Company, New York. 1976 (Chapter 5, “Weaving,” pp. 60-86).

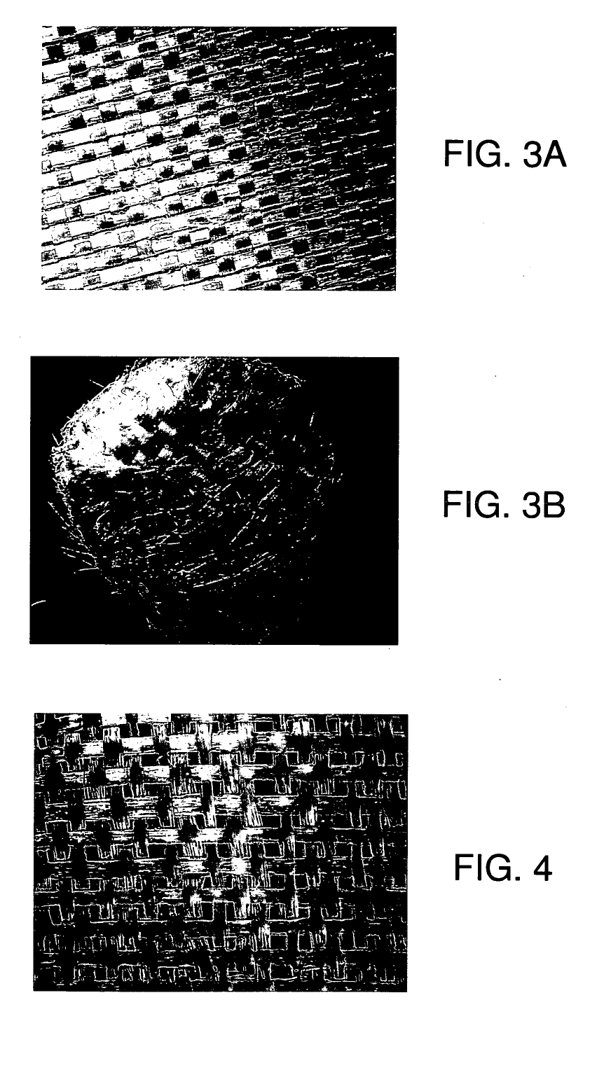

Suitable patterns for scaffold design include a simple biaxial 2-D weave (FIG. 3A) and a special Taffeta weave. In FIG. 3A, the two-dimensional fabric is woven with glass and polymer yarns.

In three-dimensional weaving, glass bundles can also be woven into almost any structures that can be woven using polymer yarns. The pore size and porosity can be controlled by varying weaving parameters. In FIG. 3B, a three-dimensional scaffold is shown. As discussed with reference to two dimensional weaving, specific procedures for different weaving patterns are numerous and widely available to those skilled in the art. An exemplary, but by no means exhaustive compilation of weaving patterns is provided in Textiles: Fiber to Fabric by M. David Potter, Bernard P. Corbman. McGraw-Hill Book Company, New York. 1976 (Chapter 5 “Weaving,” pp. 60-86).

### EXAMPLE 4

### Fabrication of Scaffolds

We will develop a bioactive, fully resorbable, synthetic three-dimensional scaffold using weaving and three-dimensional assembly methods. The scaffolds will comprise a cartilage region and a bone region. They will have different porosity and pore size for either of these two regions. Other features of the scaffold include a highly porous and lactate-rich region for promoting cartilage regeneration and a bioactive matrix that stimulates bone tissue formation and repair. Flexibility of the scaffold will be achieved by using fine and flexible bioactive glass and polymer fibers (10-25 μm diameter) and a weaving method so that the scaffold can conform to appropriate topography of cartilage to be repaired. The scaffolds will then be sterilized and used in the Example 6.

An object of Example 4 is to develop multi-region three-dimensional bioactive, resorbable and porous scaffolds. In Example 1, the rabbit study, an excellent response to the scaffold was obtained. Regardless, these scaffolds were far from ideal. Non-automated manual production made it difficult to obtain reproducible scaffolds. In addition, the scaffolds of Example 1 had low porosity (<40%). Furthermore, the scaffold made by a two-dimensional weaving method did not have a multi-region architecture and the thickness was limited by the thickness of the glass bundles. Since a high porosity allows for a better mass transfer and tissue ingrowth, it is a desirable characteristic of scaffolds for cartilage tissue repair. The scaffold to be fabricated in Example 4 will have a high porosity (>60%) for cartilage region. In addition, ease of the three-dimensional scaffold assembly process will be taken into the consideration, such that three-dimensional scaffolds with different sizes and thickness can be produced.

An objective is to develop three-dimensional scaffolds that support cartilage formation and have a reliable fixation into the defect and integration with the surrounding tissues. In addition, for defects in articular locations with substantial curvature, the scaffold should allow the tissue-engineered constructs to have appropriate topography. Other features of the scaffold include a highly porous and lactate-rich region for promoting cartilage regeneration, and a bioactive matrix that stimulates bone tissue formation and repair.

Glass fibers will be used with a composition described previously with minor modifications (Marcolongo M, Ducheyne P, LaCourse W C. (1997) Surface reaction layer formation in vitro on a bioactive glass fiber/polymeric composite. J Biomed Mater Res. 1997 Dec. 5;37(3):440-8), specifically 51% SiO₂, 29% Na₂O, 14% CaO, 6% P₂O₅ (w/w). The glass fibers will be made into glass bundles for use in the fabrication of the scaffold. Briefly, bioactive glass fibers of 15-25 μm in diameter will be coated with polylactic acid (PLA) polymer dissolved in chloroform (2% w/v) and then bound into bundles of 150-350 μm (±10%) (diameter). The PLA polymer coating serves as a binder for the glass filaments in the bundle. A PLLA polymer yarn that consists of 32-128 filaments (60-240 denier) will be used.

A biaxial weave will be made with the glass and polymer bundles. To weave the scaffold of the cartilage region, bioactive glass bundles will be used in warp direction and polymer bundles used in both warp and weft directions. The ratio of polymer/glass will be 10:90-80:20 (w/w). A reed of Dent 24-48 (24-48 bundles/inch) will be used in order to have a distance 300-450 μm between the adjacent bundles in the warp direction. By controlling the fabric feeding and moving speed, a comparable distance (300-450 μm) between the adjacent polymer bundles in weft direction will be achieved. As the size of the polymer bundles is in the range 100-150 μm, the total porosity of the resulted fabric will be considerably greater than 60%, and pore size in the range 100-500 μm.

To weave the scaffold of bone region, only bioactive glass bundles will be used in warp direction and polymer bundles in weft direction. The ratio of polymer/glass will be reduced to 20:80 (w:w) in order to have more glass content to stimulate bone formation and repair. A reed of Dent 48 will be used to create a distance 150-500 μm between the adjacent glass bundles in the warp direction. A comparable distance in the weft direction will also be achieved with polymer bundles. The pore size created in the scaffold by the process with the above setting will be in the range 100-500 μm.

One reason for having a higher PLA content in the cartilage region of the scaffold is that the degradation product of PLA, lactic acid, could promote the cells from dermal tissue to differentiate into chondrocytes. Actually the coaxing of the cells from dermal tissue towards the chondrocyte phenotype is achieved by adding lactate (and staurosporine). The possible problem that may be encountered in the weaving process is that the polymer yarn consists of multi-filaments, it might flatten out in the weave and thus reduce the pore size in the scaffold. This problem can be minimized by twisting the yarns before the weaving. It has been found that 4 twists per inch is sufficient to maintain the cylindrical shape of the polymer yarns. The pore size and porosity of the scaffold will be analyzed using light microscopy.

The woven fabrics will be folded into three-dimensional scaffolds with desired region thickness and bound together by stitching before being cut into discs of desired sizes. In the folding, the glass bundles will be placed at 90° in the adjacent layers. The discs will be cleaned in alcohol, dried in air, sterilized using γ-ray irradiation, and used for cartilage tissue formation in vitro in Example 6.

FIG. 4 provides an example of a porous scaffold which has a higher pore size and porosity than that shown in FIG. 3A.

### EXAMPLE 5

### Fabrication of Scaffolds

The objects and methods of Example 5 are the same as Example 4 with differences being that only bioactive glass fibers will be used. Briefly, bioactive glass fibers of 15-25 μm in diameter will be coated with polylactic acid (PLA) polymer dissolved in chloroform (2% w/v) and then bound into bundles of 150-350 μm (±10%) (diameter). The PLA polymer coating serves as a binder for the glass filaments in the bundle.

A biaxial weave will be made with the glass bundles. To weave the scaffolds of the cartilage and bone regions, bioactive glass bundles will be used in both the warp direction and the weft direction.

The woven fabrics will be folded into three-dimensional scaffolds with desired region thickness and bound together by stitching before being cut into discs of desired sizes. In the folding, the glass bundles will be placed at 90° in the adjacent layers. The discs will be cleaned in alcohol, dried in air, sterilized using γ-ray irradiation, and used for cartilage tissue formation in vitro in other examples.

### EXAMPLE 6

### Seeding Cells on Scaffolds,

Chondrocytes differentiated from cells isolated from human skin tissue will be cultured on the bioactive and resorbable scaffolds to form 3-D cartilage tissue in vitro. The ability of porous scaffolds to support the cellular proliferation, differentiation, and cartilage formation will be evaluated.

While not intending to be bound by theory, it is hypothesized that on a bioactive porous scaffold, cells from dermal tissue will differentiate into chondrocytes under appropriate cell culture conditions that mimic the in vivo microenvironment, such as low oxygen, a mildly acidic pH, and the presence of elevated levels of lactate. Also, the three-dimensional network of the highly porous bioactive scaffold will be conductive to three-dimensional cell-cell interaction and cartilage extracellular matrix formation. This example has two major objectives. The first objective is to determine the feasibility of converting dermal fibroblasts into chondrocyte-like cells under specifically defined in vitro cell culture conditions. The second objective is to evaluate the ability of porous bioactive glass/polymer scaffolds to support human dermal fibroblast and/or human chondrocyte proliferation, differentiation, and cartilage formation under the same in vitro cell culture conditions. Outcomes from this study will provide information as to the feasibility of using dermal fibroblasts and/or scaffolds in the subsequent phase II animal study.

Human dermal fibroblasts will be cultured on the three-dimensional scaffolds at the seeding density and in vitro cell culture conditions similar to that used for induction of fibroblasts into chondrocytes in micromass cultures (Nicoll S B, Wedrychowska A, Smith N R, Bhatnager R S (2001) Modulation of proteoglycan and collagen profiles in human dermal fibroblasts by high density micromass culture and treatment with lactic acid suggests change to a chondrogenic phenotype. Conn Tiss Res 42, 59-69). Chondrocytes will be cultured on the three-dimensional scaffolds as control. Fibroblasts cultures on the scaffolds at normal culture condition will be used for comparison purpose. Cells will be cultured for 3 weeks and will be evaluated for proliferation, differentiation and tissue formation at 1, 2, and 3 weeks. Three duplicates will be carried out for each experimental condition. The biochemical and histological evaluation methods are described below.

Cell Cultures

Human adult dermal fibroblasts are available as a Clonetics Human Cell System from Cambrex Bio Science, Walkersville, Md. Cells are supplied from a single donor and maintained in a serum-free MCDB-202 growth medium or a similar medium supplemented with 2% fetal bovine serum.

Dermal fibroblasts will be maintained in 100×20 mm culture dishes in minimum essential medium (MEM) with Earle's Balanced Salt Solution (BBS) supplemented with 25% fetal bovine serum (FBS) and 100 U/ml penicillin and 100 μg/ml streptomycin. Homogeneous, spindle-shaped fibroblasts will be expanded in MEM supplemented with 10% FBS and antibiotics in 75 cm² tissue culture flasks. Four strains from passages 3 to 8 will be used for this study. The cells will be seeded in high density cultures (2.0×10⁷ cell/ml) onto 6-mm diameter 3-mm thick porous discs. 0.5 ml cell suspension will be delivered to each disc. The cultures will be incubated for 1 hour at 37° C., 5% CO₂ to allow the cells to adhere to the scaffold. Following the incubation, the scaffolds will be flooded with MEM to bring the final volume to 2.0 ml. The protein kinase C inhibitor, staurosporine will be added to the cultures at the time of plating (50-200 nM). After an initial 24 hour period, all cells will be rinsed several times with PBS and maintained in serum-free medium. Cell cultures without the protein kinase C inhibitor and staurosporine will be used for comparison purpose. Cell adhesion, proliferation, morphology, chondrogenesis will be analyzed using histological, immunohistochemical and RT-PCR analyses.

Cell Adhesion and Proliferation

Dermal fibroblasts will be seeded (3.5×10³ cells/cm²) in 24 well tissue culture plates or on 6-mm diameter, 3 mm thick discs in either serum-free or serum-containing MCDB-202 medium. Cell number will be determined after 7 days by measuring DNA content using Hoechst 33258 dye (Molecular Probes, Eugene, Oreg.). Cell monolayers on tissue culture plastic or scaffolds will be washed in PBS, digested overnight at 37° C. in papain solution (1 mg/ml in PBS; Sigma), and then reacted with Hoechst dye (0.5/ml) in the dark for 30 min at RT. After 30 minutes, fluorescence will be quantified using a plate reader (Tecan) and concentrations of DNA determined against a standard curve made from bovine thymus DNA. Cell numbers will be calculated using the estimated value for cellular DNA content of 7.7 pg DNA/cell.

In Vitro Evaluation of Chondrogenesis

Cells will be seeded as high density micromass cultures to promote chondrogenesis. Cells in serum-containing medium are plated at 5×10⁵ cells/50 μl aliquot in 24 well plates or on 6 mm diameter prewetted scaffolds for two hours to allow cell attachment. Then 1.5 ml defined serum-free DMEM medium is added and the cultures are maintained at 37° C. in 2-5% O₂. Medium is replaced every 3-4 days with the following defined serum free medium. DMEM with ITS+Premix (insulin, transferring, selenium, linoleic acid, BSA), sodium pyruvate [100 g/ml], proline [40 g/ml], ascorbate 2-phosphate [50 g/ml], dexamethasone [10⁻⁷M], and TGF[10 ng/ml]) plus antibiotics. Human chondrocytes will serve as controls. After 7 and 21 days, the cells will be evaluated as described below. Human chondrocyte cultures will be established as a positive control cell population.

Reverse Transcription-Polymerase Chain Reaction (RT-PCR) Analysis

Total cellular RNA will be isolated by guanidinium thiocyanatephenol-chloroform extraction using the Trizol reagent (LifeTechnologies, Gaithersburg, Md.). Reverse transcription will be performed using the Superscript Preamplification System for First Strand cDNA Synthesis (Invitrogen) according to the manufacturer's instructions. The reverse transcription reaction will be carried out with oligo(dT) primers at 42° C. for 50 minutes. PCR amplification will be executed using the Advantage 2 PCR Kit (Clontech, Palo Alto, Calif.) using a 2 l sample of cDNA for each 50 l reaction. After a precycle denaturation step at 94° C. for 2 min, amplification will be performed using standard thermal cycling parameters with annealing temperatures dependent on the oligonucleotide primer set. Primer sequences for the human type II collagen, aggrecan core protein, and GAPDH (as an internal standard) were designed using a computer-aided software package based on the mRNA sequences deposited in GenBank and have been previously used by our group. The PCR products will be resolved on a 1.0% agarose gel in 1X Tris-acetate-EDTA buffer (Sigma) and visualized by ethidium bromide staining with a Kodak gel imaging system.

Histology and Immunohistochemistry

After 3 weeks in culture, the surfaces will be washed 2× with PBS and cartilage formation will be determined by hematoxylin/eosin and alcian blue staining. The identity of the attached cells will be confirmed using immunohistochemistry. Cultures will be rinsed in PBS, fixed in acid-formalin/ethanol, rinsed twice with PBS, and processed for staining with hematoxylin/eosin or monoclonal antibodies to type II collagen (Labvision, Calif.). For immunohistochemistry, the cultures will be rinsed with PBS, treated with 3% hydrogen peroxide in methanol for 10 minutes at room temperature to block endogenous peroxidase activity, rinsed with PBS, and incubated with blocking solution (10% goat serum in PBS) for 10 minutes at room temperature. The samples will then be incubated with a mouse monoclonal antibody to type II collagen (1:200 dilution in 10% goat serum in PBS) for 60 minutes at room temperature. After rinsing with PBS, cultures will be incubated with a prediluted biotin-conjugated goat-derived broad spectrum IgG secondary antibody (Zymed Laboratories, South San Francisco, Calif.) for 20 minutes at room temperature. Following a PBS rinse, the samples will be visualized using streptavidin-conjugated horseradish peroxidase and DAB as the substrate chromagen employing the Histostain-Plus kit (Zymed) as directed by the supplier. Nonimmune control specimens will be incubated with blocking solution (10% goat serum in PBS) in place of primary antibody. Cultures will be viewed with a Zeiss Stemi-2000C stereomicroscope. In addition to type II collagen immunohistochemistry, double antibody immunohistochemistry for aggrecan core protein to identify chondrocytes and vimentin to identify fibroblasts will also be performed.

By comparing the proliferation, differentiation and cartilage formation of cells from human dermal tissue and human chondrocytes cultured on the three-dimensional scaffolds, we will determine the feasibility of using dermal cells to form cartilage tissue. Specifically, if the cells from dermal tissue formed a continuous layer of tissue resembling hyaline cartilage, and the extracellular matrix produced contained proteoglycans and type II collagen macromolecules comparable to these produced in chondrocyte cultures, it will prove our hypotheses that on a bioactive porous scaffold, cells from dermal tissue will differentiate into chondrocytes under appropriate cell culture conditions, and porous bioactive scaffolds can support human dermal fibroblast and/or human chondrocyte proliferation, differentiation, and cartilage formation.

The materials of Example 5 will be also be used in a study similar to Example 6. In addition, scaffolds of Examples 6 and the like, comprising engineered cartilage tissue will be used in a sheep cartilage repair model. The resorbable nature of the scaffold is expected to result in a complete or substantially complete restoration of normal cartilage without long term presence of any cell carrier materials.

### EXAMPLE 7

### Fabrication of Scaffolds

The objective and method of this Example are the same as in Examples 3, 4, or 5 but with PLGA (poly co-lactic-glycolic acid) instead of PLA. These scaffolds are subject to a heat-treatment to enhance the coherence of the scaffolds. When 85/15 PLA/PGA is used, a 24 hour heat treatment at 80° C. leads to bonding between the PLGA polymer and therefore increases the cohesiveness of the scaffolds.

References cited herein are incorporated by reference in their entirety. Other aspects of the invention will be apparent from review of the present specification and claims and all such falling within the spirit of the invention are comprehended hereby.

1. A flexible, bioactive glass mesh comprising interwoven bioactive glass fibers coated with a resorbable polymer.

2. The mesh of claim 1 wherein said mesh comprises a porosity of between about 25% and 95%.

3. The mesh of claim 1 wherein said glass fibers are coated with a polylactic acid polymer or poly-glycolic acid polymer or both or their copolymers.

4. A flexible, bioactive mesh comprising glass fibers and first resorbable polymer fibers wherein said glass fibers are interwoven with said first resorbable polymer fibers.

5. The mesh of claim 4 wherein said glass fibers are woven perpendicularly to said first resorbable polymer fibers.

6. The mesh of claim 4 wherein said glass fibers and a first portion of said first resorbable polymer fibers are woven perpendicularly to a second portion of said first resorbable polymer fibers.

7. The mesh of claim 4 wherein the glass fibers are coated with a second resorbable polymer.

8. A flexible, bioactive scaffold comprising a plurality of bioactive meshes wherein said meshes comprise interwoven bioactive glass fibers coated with a resorbable polymer.

9. The scaffold of claim 8 wherein said plurality of bioactive meshes are laminated.

10. The scaffold of claim 8 wherein said plurality of bioactive meshes are stitched together.

11. A flexible, bioactive glass scaffold comprising a cartilage region wherein said cartilage region comprises a first bioactive mesh.

12. The scaffold of claim 11 further comprising a bone region wherein said bone region comprises a second bioactive mesh.

13. The scaffold of claim 11 wherein said first bioactive mesh comprises a porosity of between about 40% and about 95%.

14. The scaffold of claim 12 wherein said first bioactive mesh comprises a porosity of between about 40% and about 95% and said second bioactive mesh comprises a porosity of between about 25% and 80%.

15. A flexible, bioactive glass scaffold comprising a bone region wherein said bone region comprises a bioactive mesh.

16. The scaffold of claim 15 wherein said bioactive mesh comprises a porosity of between about 25% and 80%.

17. A flexible, bioactive glass scaffold comprising a non-calcified tissue region wherein said non-calcified tissue region comprises a bioactive mesh.

18. The scaffold of claim 17 wherein said bioactive mesh comprises a porosity of between about 25% and 95%.

19. A method of making a flexible, bioactive glass scaffold comprising:
pulling bioactive glass fibers;
winding said fibers;
coating said fibers with a resorbable polymer to form bundles; and
creating a biaxial weave with said bundles.

20. The method of claim 19 further comprising layering a plurality of biaxial weaves to create a three-dimensional weave.

21. The method of claim 20 wherein said plurality of biaxial weaves comprises biaxial weaves having differing porosities thereby creating a porosity gradient.

22. A method of making a flexible, bioactive glass scaffold comprising:
pulling bioactive glass fibers;
winding said fibers;
forming said fibers into bundles;
coating said bundles with a resorbable polymer; and
creating a biaxial weave with said bundles.

23. The method of claim 22 further comprising layering a plurality of biaxial weaves to create a three-dimensional weave.

24. The method of claim 23 wherein said plurality of biaxial weaves comprises biaxial weaves having differing porosities thereby creating a porosity gradient.

25. A method of engineering tissue in vitro comprising:
creating a biaxial weave comprising interwoven glass fibers;
creating a flexible bioactive glass scaffold comprising said glass fibers;
seeding fibroblasts onto said glass scaffold; and
incubating said fibroblasts.

26. A method of engineering tissue in vitro comprising:
creating a biaxial weave comprising interwoven glass fibers;
creating a flexible bioactive glass scaffold comprising said glass fibers;
seeding chondroblasts onto said glass scaffold; and
incubating said chondroblasts.

27. A method of treating a cartilage lesion in a mammal comprising:
providing a flexible, bioactive glass scaffold;
seeding chondrocyte-like cells onto said glass scaffold; and
implanting said glass scaffold into said mammal.
